(19) United States
(12) Patent Application Publication
(10) Pub. No.: US 2004/0052756 A1
(43) Pub. Date: Mar. 18, 2004

(54) Hair quality improver

(75) Inventors: Hiroyuki Taguchi, Haga-gun (JP); Mitsuyuki Hotta, Haga-gun (JP); Hideshi Kidena, Haga-gun (JP); Akira Mamada, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 10/241,694

(22) Filed: Sep. 12, 2002

(51) Int. Cl. A61K 7/06; A61K 35/78

(52) U.S. Cl. 424/74; 424/742; 424/757; 424/756; 424/769; 424/778

(57) ABSTRACT

The present invention relates a hair quality improver containing the extract, steam distillate or pressed product of a plant selected from Eucalyptus, hop, ginger, gardenia, clove or safflower.

Use of this hair quality improver brings about an improvement in the quality or touch of the hair such as strength•body, fluffiness, manageability or gloss.

### TECHNICAL FIELD

The present invention relates to a hair quality improver capable of substantially improving hair quality, thereby giving the hair flexibility, fluffiness, manageability, strength, body and gloss.

### BACKGROUND ART

Frequent repetition of beauty treatments such as shampooing, brushing, heating by a dryer, coloring or bleaching, long-time exposure to UV rays, or aging damages and deteriorates the hair. As a result, the hair becomes excessively dry and a loss of healthy gloss, an increase in the split or broken hair, or lowering in the hair strength occurs. For the purpose of retaining moisture and imparting the hair with smoothness and moist touch while overcoming the hair damage due to excessive drying, an oil component such as silicone or hydrocarbon oil, a humectant such as low molecular polyhydric alcohol or glycerin, or a raw material extracted from a natural substance, for example, protein, polysaccharide, natural polymer or a single substance or oligosaccharide constituting it such as amino acid or peptide is incorporated in hair cosmetic compositions.

When such hair cosmetic compositions are applied to the hair, however, they only cover the hair surface or replenish a target component inside of the hair so that some effects appear rightly after the application, but their durability is not sufficient.

An object of the present invention is therefore to provide a hair quality improver excellent in improvement of the very hair quality such as flexibility, fluffiness, manageability, strength, body, and gloss and in suppression of deflection.

### DISCLOSURE OF THE INVENTION

The present inventors have found that when an Eucalyptus extract is applied to the scalp to cause it to act on the hair root cells, it improves water retention of the hair, alleviates deflection, objectively improves uniformity in the hair direction (manageability) and gloss, thus substantially improving hair quality, whereby the hair feeling such as strength, body, fluffiness, manageability or gloss is improved.

An object of the present invention is therefore to provide a hair quality improver comprising, as an effective ingredient, an extract, steam distillate or pressed product of a plant selected from the group consisting of Eucalyptus, hop, ginger, gardenia, clove and safflower.

Another object of the present invention is to provide a hair quality improving method, which comprises applying, to the scalp or hair, a hair cosmetic composition containing an extract, steam distillate or pressed product of a plant selected from the group consisting of Eucalyptus, hop, ginger, gardenia, clove and safflower.

The plants for use in the present invention are known to have various medicinal properties, but their hair quality improving effects are not known.

### BRIEF DESCRIPTION OF THE DRAWINGS

FIG. 1 illustrates measurement results of water retention performance;

FIG. 2 illustrates one example of hair root lift;

FIG. 3 illustrates a difference recognition percentage according to the touch by the subject and beautician;

FIG. 4 illustrates a difference recognition percentage according to the observation by the subject and beautician;

FIG. 5 illustrates the measurement results of deflection of the hair to which an Eucalyptus extract has been applied;

FIG. 6 illustrates the measurement results of the uniformity in the direction of the hair to which an Eucalyptus extract has been applied;

FIG. 7 illustrates the measurement results of the gloss of the hair to which an Eucalyptus extract has been applied; and

FIG. 8 illustrates a curl retention percentage of perming when an Eucalyptus extract has been applied to the hair.

### THE BEST MODE FOR CARRYING OUT THE INVENTION

The Eucalyptus to be used in the present invention is Eucalyptus globulus or an allied plant thereof belonging to the family Myrtaceae. Its leaves, branches, flowers and fruit are used mainly.

The hop, Humulus Lupulus, is a plant of the family Moraceae and its male flower spike is mainly employed.

The ginger, Zingiber officinale Roscoe, is a plant of the family Zingiberaceae and its rhizome (Zingiberis Rhizoma) is mainly employed.

The gardenia, Gardenia jasminoides Ellis, is a plant of the family Rubiaseae and its fruit is mainly employed.

The clove, Syzygium aromaticum Merrill et Perry (Eugenia caryophyllata Thunberg) is a plant of the family the Myrtaceae and its bud, flower stalk, unripened fruit or leaves are mainly employed.

The safflower, Carthamus tinctorius Linne, is a plant of the family Compositae and its flower or a portion thereof from which most of a yellow pigment has been removed, or whole plant is employed.

Of these plants, Eucalyptus, ginger and hop are especially preferred. These plants may be used in combination, or two or more of the extracts, steam distillates or pressed products of these plants may be used as a mixture. In this case, a combined use of Eucalyptus and ginger or Eucalyptus and hop is preferred.

In the present invention, the extract, steam distillate or pressed product of a plant such as Eucalyptus can be obtained from the plant itself or a dried and pulverized one. A more purified plant such as essential oil, or a commercially available product is also usable. A portion of the plant subjected to extraction, steam distillation or pressing is preferably a leaf.

As the solvent to be employed upon extraction, those ordinarily employed for the extraction of a plant component are usable. Examples include water, petroleum ether, n-hexane, toluene, dichloroethane, chloroform, ether, ethyl acetate, acetone, methanol, ethanol, propanol, butanol, ethylene glycol, propylene glycol and butylene glycol. Of these, water, ethanol, propylene glycol and butylene glycol are preferred. They may be used either singly or in combination. Extraction can be performed under ordinary extracting conditions. For example, the above-described plant may be immersed in the solvent or heated under reflux at 3 to 100° C. for several hours to several weeks. When the plant is used as an essential oil, it can be prepared by the ordinary method, for example, by subjecting the plant to steam distillation, extraction or pressing. Although the extract, steam distillate or pressed product of a plant such as Eucalyptus can be used as it is as an effective ingredient of the hair quality improver of the present invention, a highly active fraction obtained by a more proper separating means such as gel filtration, chromatography or precision distillation can also be used.

As will be described later in Examples, the plant extract or the like of the present invention thus prepared, when applied to the hair, improves water retention performance, lessens deflection, and objectively improves uniformity in the hair direction (manageability) and gloss, thereby substantially improving the hair quality itself.

The extract, steam distillate or pressed product of a plant such as Eucalyptus can be applied directly to the scalp or hair after diluting it with a solvent ordinarily employed for extraction, preferably water, ethanol, propylene glycol, butylene glycol, a mixed solvent of water and ethanol, a mixed solvent of water and propylene glycol, or a mixed solvent of water and butylene glycol. Incorporation of it usually in an amount of 0.000001 to 10 wt. %, especially 0.00001 to 5 wt. % in terms of a solid content is preferred.

The hair quality improver of the present invention may be added to a hair cosmetic composition. Examples of the components to be incorporated in the hair cosmetic composition include surfactants such as anionic surfactants, cationic surfactants, nonionic surfactants and amphoteric surfactants, silicones, hydrocarbons, higher alcohols, lanoline derivatives, higher fatty acid esters, higher fatty acids, oils or fats, glycerin, betaines, humectants, cationic polymers, polysaccharides, polypeptides, pearling agents, solvents, liquid-crystal forming bases, aromatic sulfonic acids, colorants, perfumes, propellants, hair quality improvers other than those of the present invention, hair growth accelerators, chelating agents, pH regulators and antidandruffs.

Hair cosmetic compositions containing the hair quality improver of the present invention can be provided in the desired form, for example, an aqueous solution, ethanol solution, emulsion, suspension, gel, liquid crystal, solid or aerosol. Specific examples include hair rinse, hair conditioner, hair treatment, hair lotion, hair pack, hair cream, conditioning mousse, hair mousse, hair spray, shampoo, and leave-on treatment.

### EXAMPLES

### Preparation Example

Preparation of Eucalyptus Extract

Leaves of Eucalptus globulus Labillardiere were cut into thin pieces and to 10 g of them, 100 mL of a mixed solvent (20:80) of water and 1,3-butanediol was added. At room temperature, extraction from the mixture was conducted for 24 hours while occasionally stirring, followed by filtration. The filtrate was matured by allowing it to stand at 5° C. for 7 days and, from the matured filtrate, the grout and precipitate were filtered off. Water (100 mL) was added to the residue, followed by concentration at 40° C. under reduced pressure to about 70 mL. After conducting the above-described operation 3 times, water and 1,3butanediol were added, whereby an extract adjusted to have a total quantity of 100 mL and a 1,3-butanediol concentration of 80 v/v% was obtained (solid content: 0.3 wt. %).

In a similar manner to that employed for the preparation of the Eucalyptus extract except for the use of the male flower strike of hop (Humulus lupulus Linne) and the fruit of gardenia (Gardenia jasminoides Ellis) as the plant and 1,3-butanediol as the solvent, and except for the use of the rhizome of ginger (Zingiber officinale Roscoe), buds of clove (Syzygium aromaticum Merrilet Perry) and the whole plant of safflower (Carthamus tinctorius Linne) as the plant and a mixed solvent (50:50) of water and ethanol as the solvent, hop, gardenia, ginger, clove and safflower extracts were prepared, respectively.

### Example 1

Water retention property was analyzed by the following evaluation method.

The dorsal hair of each of ten 7-weeks-old white guinea pigs (♂) was shaved by hair clippers and an area of 3×7 cm was marked on both the right and left sides of the back as application sites of the following samples.

| Invention product | | Control | |
|---|---|---|---|
| Eucalyptus extract | 3% | Ethanol | 60% |
| Ethanol | 60% | Water | 40% |
| Water | 37% | | |

The invention product and control product were applied to the right side and the left side of the back respectively, in an amount of 0.3 mL once a day from the shaving day.

On Day 4, the dorsal hair was shaved again and the application was continued for further 10 straight days.

After completion of the application, the guinea pigs were sacrificed and the dorsal skin collected from them was precisely cut into a piece of 3×6 cm within the application site of the sample. The regrowth hair was shaved and collected.

Measurement of the Water Content of the Dorsal Hair

The hair thus collected was washed with 20 mL of distilled water for 20 minutes, washed with 10 mL of hexane for 2 minutes to remove the stain from the hair surface, rinsed lightly with distilled water and then, air dried.

After the hair was allowed to stand for 10 days at temperature of 23° C. and relative humidity of 0%, it was weighed precisely. Precise weighing was conducted again after the hair was allowed to stand for 14 days at temperature of 23° C. and relative humidity of 45%.

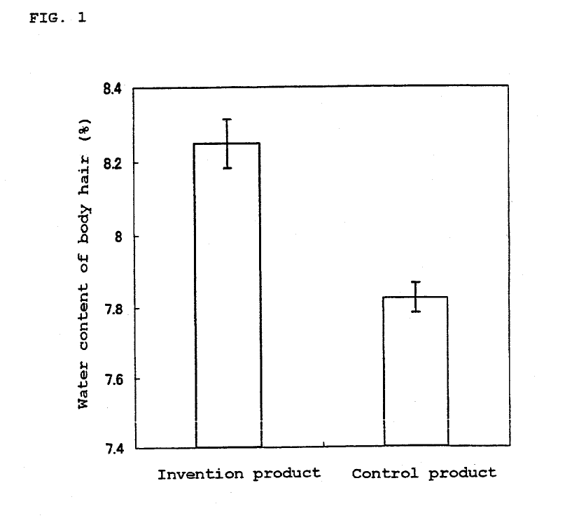

A difference obtained by subtracting the hair weight at relative humidity of 0% from the hair weight at relative humidity of 45% is designated as a hair water content at 23° C. and relative humidity of 45%. The measurement results are shown in FIG. 1.

The hair which appeared newly after the application of the invention product was found to exhibit excellent water retention.

### Example 2

Eleven females and one male, who were healthy and on the 30's, were asked to apply, to the halves of their scalp, two aerosol preparations described below, respectively, twice a day. For 6 months after the test was started, they were prohibited from permanent weaving and hair dyeing and asked to use a predetermined shampoo which was commercially available and popular (6 months after the test, three were subjected to permanent weaving and two were hair-dyed by a beautician determined in advance). The touch of the hair was evaluated by both the same beautician and the subject according to double-blind evaluation so that they would not know which preparation was applied.

Evaluation was conducted by the same beautician after shampooing.

Formulation 1) filled, as a 2.2% CO₂ gas aerosol, with 3% of Eucalyptus extract, 50% of 95% ethanol, and a balance of desalted water.

Formulation 2) filled, as a 2.2% CO₂ gas aerosol, with 50% of 95% ethanol and a balance of desalted water.

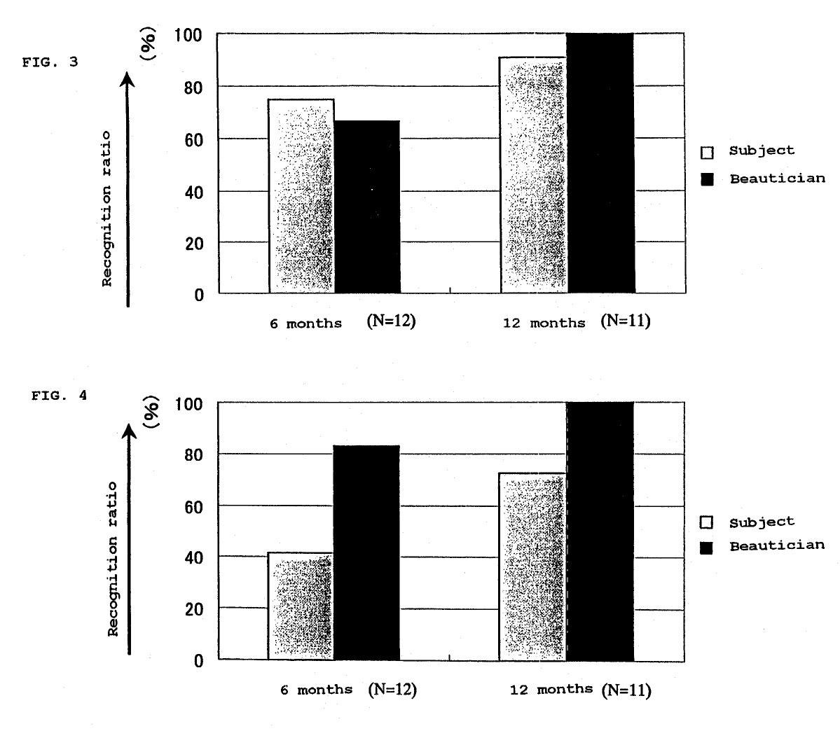

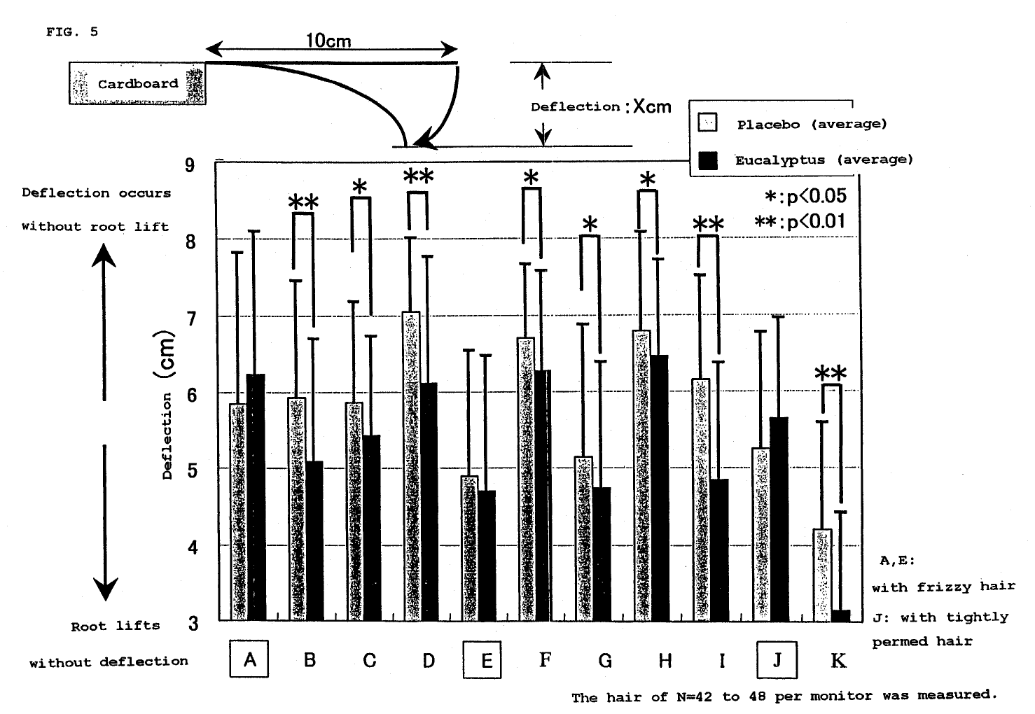

As a result, from about 3 months after the application, a marked root lift of the hair was visually observed on the application site of the Formulation 1 and from about 6 months after the application, comments as shown in (Table 1) were offered not only by the beautician but also the subject. In FIGS. 3 and 4, a difference recognition percentage according to the touch to the right and left sides and that according to the observation are shown, respectively, by the percentage of the persons who have recognized the difference. After 12 months or so, 90% or more of the subjects recognized the difference in touch and 70% or more recognized the difference in observation results, suggesting that the difference was great. With regards to the difference between the right side and left side, the side to which the formulation 1 was applied received positive evaluation and was preferred.

TABLE 1
Comment 6 months after the application was started

| Side to which Eucalyptus extract | | | |
|---|---|---|---|
| was applied | | Placebo side | |
| Subject | Beautician | Subject | Beautician |
| Fluffy | Fluffy | Having neither | Not smooth |
| Having strength and | Smooth | strength nor | Having much |
| body | Moisturized | body and weak | flyway hair |
| Elastic at the hair root | (like the hair of | Not uniform at | Poor in |
| Imparted with gloss | children) | hair end | moisture |
| Manageable | Uniform in the | Lacking in | Not uniform |
| Uniform at hair end | hair direction | strength | in the hair |
| Having gloss and | Hair root seems | Lacking in | direction |
| moistured touch | to lifted up | body | |
| Each hair seems to be | Improved in | | |
| thickened | thickness and | | |
| Having strength and | volume | | |
| softness | Smooth finger | | |
| | styling | | |

### Example 3

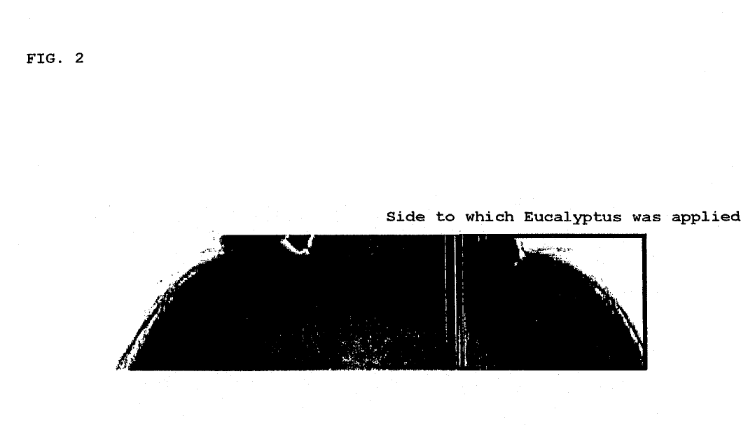

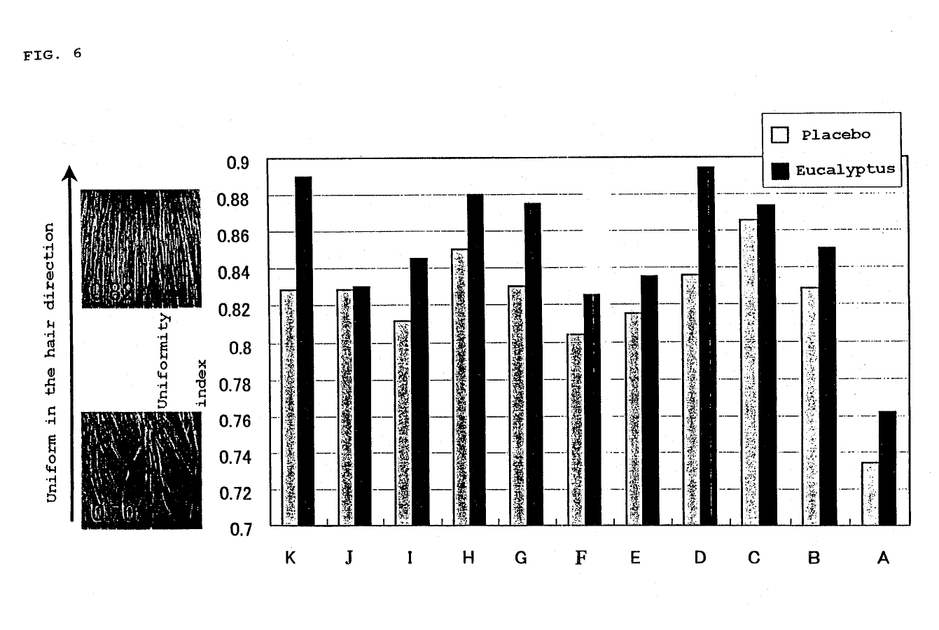

Upon completion of the test in Example 1, the hair was collected without removing its root and its “deflection” was measured. A hair bundle of 12 cm (about n=45) was attached to a cardboard with an overlap width of 2 cm. It was allowed to stand at 20° C. and RH of 65% for 72 hours. The distance from the cardboard to the position of the hair after sagging (×cm in FIG. 5) at 25° C. and 65% RH was measured as the amount of “deflection”. The amount of “deflection” can be determined easily by measuring how much the hair sagged under its own weight and this indicates the resilience of the hair. As a result, since the deflection amount on the side to which the formulation 1) had been applied showed a decrease, a root lift of the hair, which had been confirmed visually (FIG. 2), was also confirmed quantitatively by this evaluation method.

### Example 4

An improvement in the “uniformity of the hair direction (manageability)” of the hair was visually apparent, but its “uniformity” was measured by taking the picture of the hair at the test in Example 1. Described specifically, the picture of the hair of “thickness: about 1 cm×width: about 10 cm” was taken by a digital camera (5000000 pixels). To each pixel of a luminance signal, Prewitt operator (Computer vision and image processing: a practical approach using CVIP tools/Scott E Umbaugh/p64-65/Prentice-Hall, Inc) was applied. The vertical direction component p₁ and horizontal direction component p₂ were subjected to principal component analysis to obtain dispersion σ₁² on the first main axis and dispersion σ₂² on the second main axis, from which a uniformity index 1-σ₂/σ₁ was determined. As a result, improvement in the uniformity of the hair direction on the side to which the formulation 1) had been applied was apparent not only visually but also quantitatively (FIG. 6).

### Example 5

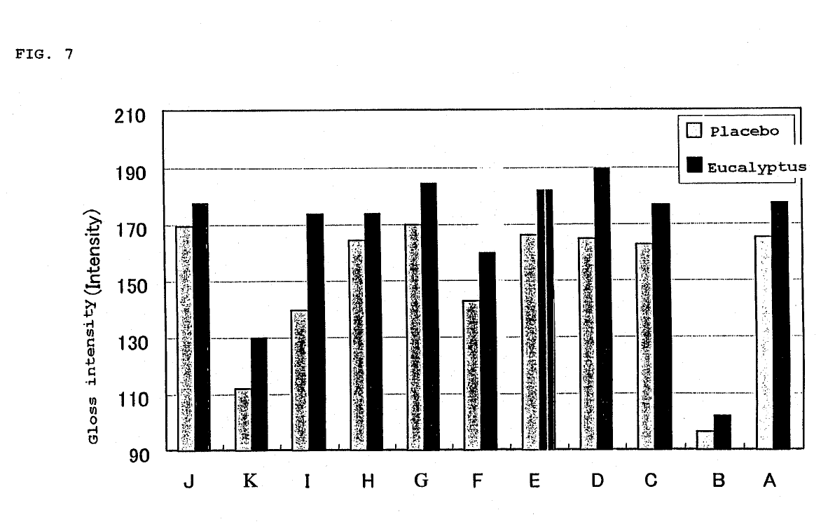

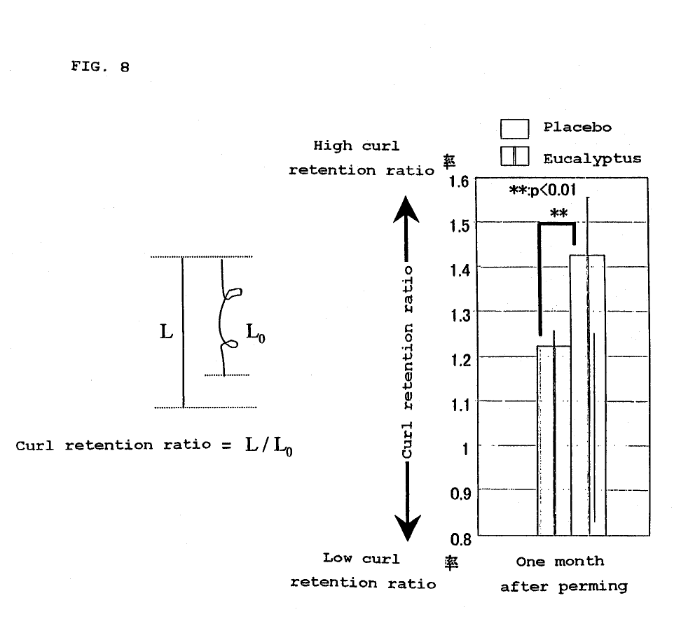

An improvement in the “gloss” of the hair was apparent, but the “gloss” of the hair upon completion of the test in Example 1 was measured. Described specifically, the gloss of the hair was measured in accordance with the method described in Japanese Patent Laid-Open No. 2000-206044. As a result, an improvement of the gloss of the hair on the side to which the formulation 1) had been applied was indicated not only visually but also quantitatively (FIG. 7).

### Example 6

A curl retention ratio (the hair length under the natural state after permanent waving/the length of the same hair when it was pulled) of the hair of each of the monitors subjected to permanent waving treatment on Month 9 of the test in Example 1 was measured (FIG. 8).

The monitors after the test made a comment that “the hair has lost its gloss”, “the hair has become bristle and its curls are not uniform” on the side to which the formulation 2) has been applied, while “the hair has excellent gloss”, “the hair is fluffy and has strength”, or “the curls are made manageable” on the side to which the formulation 1) has been applied. Thus, an improvement in the permanent wave retention ratio on the side to which the formulation 1) had been applied was apparent from the comment.

From the above-described results, it has been apparent that application of a lotion containing an Eucalyptus extract to the hair not only brings about improvements in the touch of the hair, such as strength, body, fluffiness, manageability, uniformity of the hair direction and gloss but also exhibits quantitative improvements in the “deflection”, “uniformity of the hair direction (manageability)” and “gloss”. Thus, the invention product has marked effects on the improvement of the hair quality. The reflection of the hair becomes more stereoscopic and sharp owing to the uniform hair direction and improved gloss, which is presumed to lead to a visual improvement.

### Example 7

A hair lotion having the below-described composition can be prepared in a manner known per se in the art.

This hair lotion is excellent in water retention performance and imparts the hair with strength, body, smoothness and moisturized feeling.

TABLE 2

| Component | | | | | | Wt. % |
|---|---|---|---|---|---|---|
| No. | 1 | 2 | 3 | 4 | 5 | 6 |
| Ethanol | 60.0 | 60.0 | 60.0 | 60.0 | 60.0 | 60.0 |
| Propylene | 2.0 | 2.0 | 2.0 | 2.0 | 2.0 | 2.0 |
| glycol | | | | | | |
| Methylparaben | 0.1 | 0.1 | 0.1 | 0.1 | 0,1 | 0.1 |
| Perfume | 0.5 | 0.5 | 0.5 | 0.5 | 0.5 | 0.5 |
| Eucalyptus | 3.0 | — | — | — | — | — |
| extract | | | | | | |
| Hop extract | — | 3.0 | — | — | — | — |
| Ginger extract | — | — | 3.0 | — | — | — |
| Gardenia | — | — | — | 3.0 | — | — |
| extract | | | | | | |
| Clove extract | — | — | — | — | 3.0 | — |
| Safflower | — | — | — | — | — | 3.0 |
| extract | | | | | | |
| Purified water | Balance | Balance | Balance | Balance | Balance | Balance |
| Total | 100.0 | 100.0 | 100.0 | 100.0 | 100.0 | 100.0 |

### Example 8

A hair treatment having the below-described composition can be prepared in a manner known per se in the art.

This hair treatment is excellent in water retention performance and imparts the hair with strength, body, smoothness and moisturized feeling.

TABLE 3

| Component | | | | | | Wt. % |
|---|---|---|---|---|---|---|
| No. | 7 | 8 | 9 | 10 | 11 | 12 |
| Behenyl- | 2.0 | 2.0 | 2.0 | 2.0 | 2.0 | 2.0 |
| trimethyl | | | | | | |
| ammonium | | | | | | |
| chloride | | | | | | |
| Cetylstearyl | 5.0 | 5.0 | 5.0 | 5.0 | 5.0 | 5.0 |
| alcohol | | | | | | |
| Glycine | 1.0 | 1.0 | 1.0 | 1.0 | 1.0 | 1.0 |
| Methyl paraben | 0.1 | 0.1 | 0.1 | 0.1 | 0.1 | 0.1 |
| Perfume | 0.5 | 0.5 | 0.5 | 0.5 | 0.5 | 0.5 |
| Eucalyptus | 3.0 | — | — | — | — | — |
| extract | | | | | | |
| Hop extract | — | 3.0 | — | — | — | — |
| Ginger extract | — | — | 3.0 | — | — | — |
| Gardenia | — | — | — | 3.0 | — | — |
| extract | | | | | | |
| Clove extract | — | — | — | — | 3.0 | — |
| Saff lower | — | — | — | — | — | 3.0 |
| extract | | | | | | |
| Purified water | Balance | Balance | Balance | Balance | Balance | Balance |
| Total | 100.0 | 100.0 | 100.0 | 100.0 | 100.0 | 100.0 |

### INDUSTRIAL APPLICABILITY

Use of the hair quality improver of the present invention improves water retention of the hair and at the same time, lessens deflection, and objectively improves uniformity of the hair direction (manageability) and gloss, thereby substantially improving the hair quality, whereby the quality or touch of the hair such as strength, body, fluffiness, manageability and gloss can be improved.

1. A hair quality improver comprising an extract, steam distillate or stressed product of a plant selected from the group consisting of Eucalyptus, hop, ginger, gardenia, clove and safflower.

2. A hair quality improver according to claim 1, for improving the water retention of hair, manageability or gloss of the hair, or lessens deflection of the hair.

3. A hair quality improver according to claim 1 or 2 for improving the flexibility, fluffiness, manageability, strength, body or gloss of the hair.

4. A hair quality improving method, which comprises applying, to the scalp or hair, a hair cosmetic composition containing an extract, steam distillate or pressed product of a plant selected from the group consisting of Eucalyptus, hop, ginger, gardenia, clove and safflower.
